(19) United States
(12) Patent Application Publication
(10) Pub. No.: US 2015/0293200 A1
(43) Pub. Date: Oct. 15, 2015

(54) Methods for Generating Imaging Biomarkers Based on Diffusion Tensor Imaging of the Spinal Cord

(71) Applicant: The Medical College of Wisconsin, Inc., Milwaukee, WI (US)

(72) Inventors: Shekar N. Kurpad, Milwaukee, WI (US); Aditya Vedantam, Milwaukee, WI (US); Michael B. Jirjis, Milwaukee, WI (US); Brian D. Schmit, Milwaukee, WI (US); John L. Ulmer, Milwaukee, WI (US)

(21) Appl. No.: 14/684,997

(22) Filed: Apr. 13, 2015

Related U.S. Application Data
(60) Provisional application No. 61/978,587, filed on Apr. 11, 2014.

(51) Int. Cl. G01R 33/563; A61B 5/00; A61B 5/055

(52) U.S. Cl. CPC G01R 33/56341; A61B 5/055; A61B 5/4064; A61B 5/407

(57) ABSTRACT

Systems and methods for producing imaging biomarkers that indicate a tissue state in the nervous system of a subject based on diffusion tensor imaging (“DTI”) of the subject's spinal cord or brain are provided. The imaging biomarker can indicate a tissue state in the central nervous system (e.g., brain or spinal cord) of a subject based on DTI of the subject's spinal cord. As another example, however, the imaging biomarker can indicate a tissue state in the subject's spinal cord based on DTI of the subject's brain. The imaging biomarker is also capable of determining an efficacy of a treatment administered to a subject's spinal cord.

### CROSS-REFERENCE TO RELATED APPLICATIONS

This application claims the benefit of U.S. Provisional Patent Application Ser. No. 61/978,587, filed on Apr. 11, 2014, and entitled “METHODS FOR GENERATING IMAGING BIOMARKERS BASED ON DIFFUSION TENSOR IMAGING OF THE SPINAL CORD.”

### STATEMENT REGARDING FEDERALLY SPONSORED RESEARCH

This invention was made with government support under I01 RX000113-01 awarded by the Department of Veterans Affairs. The government has certain rights in the invention.

### BACKGROUND OF THE INVENTION

The field of the invention is systems and methods for magnetic resonance imaging (“MRI”). More particularly, the invention relates to systems and methods for producing imaging biomarkers based on diffusion tensor imaging (“DTI”) of the spinal cord.

Current medical technology is still unable to accurately predict the prognostic and diagnostic outcome after a spinal cord injury (“SCI”). Following an incomplete SCI, between 50 and 75 percent of patients will typically regain some ambulatory function. While patients with incomplete SCI are able to regain some loss of function, this seemingly random chance of recovery is in part due to the immediate physiological response that masks the true extent of the injury.

Current initial diagnosis of SCI is reliant upon anatomical medical imaging scans and rudimentary functional testing of both the motor and sensory pathways. These limited views of the spinal cord could exhibit complete SCI when in reality the spinal cord damage is minimal after inflammation is reduced and it is also possible that a normal looking spinal cord could be microscopically damaged upon further investigation. It is important to fully understand the complexity of a spinal cord injury before being able to provide a way to recovery from the disability. Physicians are able to offer some insight into the initial diagnosis of spinal cord injury, however extent of injury is hard to predict because of the initial inflammatory response.

This acute inflammatory response caused by mechanical trauma to the blood vessels and cellular structures is characterized by edema and an increase of plasma fluid into extracellular space. This increase results in a pressure induced ischemia where diminished blood flow to the injured region ensues. There is also a cytotoxic response that results in intracellular swelling.

Following the initial traumatic injury and inflammatory response, secondary damage starts to take place in the spinal cord. The injured axons close off and create bulbs that swell and progressively retract. Early after SCI, the myelin will start to break down and an influx of macrophages will take away the loose cellular structure.

The extensive damage that occurs throughout entire axons following neuronal damage in the spinal cord is not limited to the spinal cord. A number of studies have demonstrated cortical changes as a result of spinal cord injury. This cortical reorganization is not limited to the short term but has also been expressed long-term.

Thus, there is a need to provide a non-invasive diagnostic tool that is capable of monitoring changes in cortical structure that occur after SCI, or as a result of other pathological changes in the spinal cord (e.g., as a result of neurodegenerative diseases).

Similarly, studies have shown that injury to the brain can result in structural changes to the spinal cord. For instance, following a traumatic brain injury, diminished motor and sensory function can occur in associated regions of the body. As an example, damage to the motor cortex can result in Wallerian degeneration that extends down through the spinal cord.

Thus, there is a need to provide a non-invasive diagnostic tool that is capable of monitoring the progression of an injury without the need to excise tissue. For example, there remains a need to provide a diagnostic tool that will allow clinicians to assess the extent of an injury to the central nervous system that originates from an injury to the brain, or from other pathological changes occurring in the brain. There also remains a need to provide a diagnostic tool that will allow clinicians to identify the regions associated with an injury to the central nervous system that has progressed from an initial injury site. Having this information will allow clinicians to better design treatment plans.

Diffusion tensor imaging (“DTI”) is a magnetic resonance imaging (“MRI”) method that can non-invasively map the diffusion of molecules (e.g., water molecules) in living biological tissues. This diffusion can be represented mathematically as a tensor (i.e., the diffusion tensor). Because of the unique cellular and structural properties of the brain and spinal cord, DTI is particularly well-suited for revealing microscopic details about the brain and spinal cord.

Although DTI is now commonly used to study brain pathology, it has yet to be adopted for studying spinal cord pathology. As a result, most currently available commercial DTI software is primarily designed to process and evaluate brain images. Because the anatomy of the spinal cord is distinct from the brain, it is important for clinicians, and spinal cord researchers, to have software designed specifically to process and evaluate spinal cord diffusion tensor images. With the increasing use of spinal cord DTI to study spinal cord disorders, there is a clear need for such software.

### SUMMARY OF THE INVENTION

The present invention overcomes the aforementioned drawbacks by providing systems and methods for producing imaging biomarkers that indicate a tissue state in the nervous system of a subject based on diffusion tensor imaging (“DTI”) of the subject's spinal cord or brain.

It is an aspect of the invention to provide a method for producing an imaging biomarker that indicates a tissue state at a spinal cord level using an MRI system. Diffusion tensor imaging data acquired from a spinal cord of a subject with an MRI system is provided. A diffusion metric map at a first spinal cord level is produced from the provided diffusion tensor imaging data, wherein the diffusion metric map contains diffusion metric values associated with locations in the first spinal cord level. Based on the produced diffusion metric map at the first spinal cord level, a tissue state for tissues at a second spinal cord level that is distal to the first spinal cord level is determined. The second spinal cord level may be rostral to the first spinal cord level, or the second spinal cord level may be caudal to the first spinal cord level.

It is another aspect of the invention to provide a method for producing an imaging biomarker that indicates a tissue state at locations in a brain of a subject using an MRI system. Diffusion tensor imaging data acquired from a spinal cord of a subject with an MRI system is provided. A diffusion metric map at a spinal cord level is produced from the provided diffusion tensor imaging data, wherein the diffusion metric map contains diffusion metric values associated with locations in the spinal cord level. Based on the produced diffusion metric map at the spinal cord level, a tissue state for tissues in a brain of the subject is determined.

It is yet another aspect of the invention to provide a method for producing an imaging biomarker that indicates a tissue state at a spinal cord level using an MRI system. Diffusion tensor imaging data acquired from a brain of a subject with an MRI system is provided. A diffusion metric map at a location in the subject's brain is produced from the provided diffusion tensor imaging data, wherein the diffusion metric map contains diffusion metric values associated with locations in the subject's brain. Based on the produced diffusion metric map at the location in the subject's brain, a tissue state for tissues at the spinal cord level in the subject's spinal cord is determined.

It is yet another aspect of the invention to provide a method for producing an imaging biomarker that indicates an efficacy of a treatment delivered to a spinal cord of a subject. Diffusion tensor imaging data acquired from a location in a central nervous system (CNS) of a subject with an MRI system is provided. A diffusion metric map at the first location from the provided diffusion tensor imaging data, wherein the diffusion metric map contains diffusion metric values associated with locations in the subject's CNS. Based on the produced diffusion metric map at the location in the subject's CNS, an efficacy of a spinal cord treatment at a spinal cord level in the subject's spinal cord is determined. The location in the subject's CNS can be a location in the subject's brain, or in the subject's spinal cord.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of an example method for producing an imaging biomarker that indicates a tissue state at a spinal cord level distal to a spinal cord level at which diffusion metrics are computed;

FIG. 2 illustrates a comparison between severity groups rostral and caudal to an injury site for FA, MD, lADC, and tADC;

FIG. 3 illustrates average MD in the cervical segments of the spinal cord, and shows that the average MD over the cervical segments of the spinal cord for different injury severity groups (e.g., mild, moderate, and severe injuries) can be significantly different than a normal control (p<0.05);

FIG. 4 illustrates white matter and gray matter differences between injury severity groups;

FIG. 5 is a flowchart setting forth the steps of an example method for producing an imaging biomarker that indicates a tissue state at a location in a subject's brain based on diffusion metrics computed at a location in the subject's spinal cord;

FIG. 6 is a flowchart setting forth the steps of an example method for producing an imaging biomarker that indicates a tissue state at a spinal cord level in a subject's spinal cord based on diffusion metrics computed at a location in the subject's brain;

FIG. 7 is a flowchart setting forth the steps of an example method for producing an imaging biomarker that indicates an efficacy of a treatment delivered to a subject's central nervous system based on diffusion metrics computed at a location in the subject's spinal cord;

FIG. 8 illustrates in vivo diffusion metric values for 2 weeks, 5 weeks, and 10 weeks post neural stem cell transplant treatment as well as ex vivo diffusion metric values, where all values were measured in region-of-interest selections over the entire spinal cord in a rat study;

FIG. 9 is a block diagram of an example of a magnetic resonance imaging (“MRI”) system;

FIG. 10 illustrates a scatter plot showing correlation between fractional anisotropy (“FA”) at the level of maximum cord compression and change in the mJOA clinical score at three months after decompressive surgery and in 27 patients with cervical spondylotic myelopathy;

FIGS. 11A-11C illustrate average FA values at different locations along the neural axis (e.g., AICFA=anterior internal capsule fractional anisotropy; C12 and/or T6 FA=fractional anisotropy at C12 or T6 respectively; LMCFA=level of maximum compression fractional anisotropy) and compare these FA values between control and patient groups; and

FIG. 12 is a block diagram of an example computer system that can be configured to implement the methods described herein

### DETAILED DESCRIPTION OF THE INVENTION

Described here are systems and methods for producing imaging biomarkers that indicate a tissue state in the nervous system of a subject based on diffusion tensor imaging (“DTI”) of the subject's spinal cord. As an example, the imaging biomarker can indicate a tissue state in the central nervous system (e.g., brain or spinal cord) of a subject based on DTI of the subject's spinal cord. As another example, however, the imaging biomarker can indicate a tissue state in the subject's spinal cord based on DTI of the subject's brain.

Example indications of a tissue state include an indication of whether tissues at a particular location are injured or otherwise affected by pathological changes. As an example, injury can include trauma or an inflammatory response. Pathological changes can be those changes that occur as a result of pathological processes, including those attributable to pathologies such as multiple sclerosis, amyotrophic lateral sclerosis, cervical myelopathy, stroke of the spinal cord, spinal cord hemisections, radiculopathy, and spinal cord tumors.

As described below, DTI can be used as a diagnostic and prognostic tool in understanding the changes that occur throughout the spinal cord and brain following a spinal cord injury (“SCI”), or as a result of pathological changes to the central nervous system, and following a treatment administered to the central nervous system, such as a stem cell transplant.

The diffusion of water inside the nervous system is dramatically altered around the lesion site following a traumatic SCI. Following damage to the spinal cord, little is known about the diffusion characteristics of tissues located away from an injury and even less is understood about DTI's sensitivity to structural changes that occur following regenerative transplant therapies. The systems and methods described here provide diagnostic and prognostic indicators of changes in a tissue state in the central nervous system at a location that is remote from an injury site, or at a location where pathological changes are occurring or have occurred. Thus, the systems and methods described here can provide clinicians with a method for tracking and monitoring the progression of injury, the progression of central nervous system pathologies, and the effectiveness of a treatment regime, such as stem cell transplants.

It is a discovery of the invention that DTI is sensitive to changes in tissue structure in regions remote from injury to, pathological changes in, and treatment of the central nervous system. As one example, mean water diffusion in the distal locations of the spinal cord and in the brain may decrease following SCI. As another example, neuronal stem cells that are known to elicit astrocytic proliferation can produce mean increases in water diffusion.

In some embodiments, the imaging biomarker can be derived from a diffusion metric map at a first spinal cord level, and can indicate a tissue state for tissues at a second spinal cord location that is different from the first spinal cord location. As an example, the second spinal cord location can be caudal to the first spinal cord location. As another example, the second spinal cord location can be rostral to the first spinal cord location. The imaging biomarker can thus indicate an injury to tissues at the second spinal cord level based on the diffusion metrics computed at the first spinal cord level. Such an imaging biomarker is advantageous for instances where the second spinal cord level cannot be directly imaged. It may not be possible to directly image the second spinal cord level when trauma to that location, or when a surgical implant such as a spinal fixation apparatus, confounds magnetic resonance imaging of the tissues at that location.

As one example, it is a discovery of the invention that DTI of the high cervical cord can be used to diagnose injuries and pathologies in the spinal cord well removed from the imaged region in the high cervical cord. As described below, diffusion metrics that are computed from DTI data can be used as imaging biomarkers that indicate changes in areas of the spinal cord that are remote from the injured or otherwise pathological site. These diffusion metrics correlate with both histological and functional metrics of the spinal cord (i.e., the “gold standards” used to assess spinal cord structure and function); thus, the diffusion metrics provide an accurate and actionable imaging biomarker for clinical decision-making related to injury or multiple different pathologies of the spinal cord.

Not only can the imaging biomarkers derived from DTI data of the spinal cord indicate the tissue state of tissues located at a spinal cord level different from the spinal cord level at which the diffusion metrics are computed, the imaging biomarkers can indicate a severity of the injury or pathology at that remote spinal cord level.

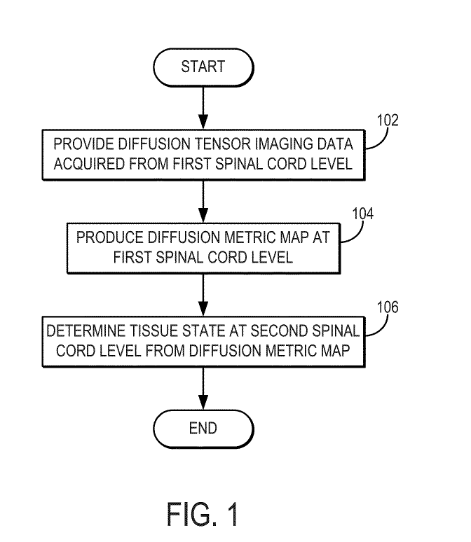

Referring now to FIG. 1, a flowchart setting forth the steps of an example method for producing an imaging biomarker that indicates a tissue state at a spinal cord level distal to a spinal cord level at which diffusion metrics are computed is illustrated.

The method includes providing DTI data that has been acquired from the subject's spinal cord with an MRI system, as indicated at step 102. As an example, the DTI data can be provided by retrieving previously acquired data from a storage device or database, or by acquiring the data using an MRI system. For the latter, the DTI data can be acquired by directing the MRI system to perform a suitable diffusion-weighted pulse sequence that is configured to acquire sufficient data for DTI. As an example, diffusion-weighted images can be acquired for six different diffusion encoding directions using a diffusion-weighted spin echo pulse sequence using b-values of 0 s/mm² for the reference image and a b-value of 500 s/mm² for the diffusion-weighted images.

The provided DTI data is then processed to produce at least one diffusion metric map at a first spinal cord level, as indicated at step 104. This processing may include pre-processing steps, such as reorienting the diffusion-weighted image, and removing artifacts and distortions from the images. For example, the diffusion-weighted images can be co-registered to correct for-eddy current and susceptibility distortions.

Processing the DTI data includes computing a diffusion tensor for voxels associated with locations in the first spinal cord location based on the DTI data acquired from those locations. From each computed diffusion tensor, one or more diffusion metrics can be computed, which can then be used to produce maps of each diffusion metric for the first spinal cord level. As an example, the diffusion metric can be the longitudinal apparent diffusion coefficient (“lADC”), which is represented by the principal eigenvalue, λ₁, of the diffusion tensor; the transverse apparent diffusion coefficient (“tADC”), which can be calculated as the mean of the secondary and tertiary eigenvalues, λ₂ and λ₃, of the diffusion tensor; the mean diffusivity (“MD”), which can be calculated as the trace of the diffusion tensor; and the fractional anisotropy (“FA”), which represents the overall anisotropy of diffusion at a certain voxel.

Based on the one or more diffusion metric maps produced at the first spinal cord level, an imaging biomarker that indicates a tissue state for tissues at a second spinal cord level is generated, as indicated at step 106. In general, the imaging biomarker is based on a statistical analysis of diffusion metrics at the first spinal cord level. For instance, the diffusion metrics can be compared with established normal values and the comparison can be used to establish a correlation with a tissue state in the second spinal cord level.

In some embodiments, image analysis of the diffusion metric maps can be performed based on one or more regions-of-interest (“ROIs”) selected or otherwise identified in the diffusion metric maps. For instance, a user can manually draw an ROI on the diffusion metric map, or an automated process can be performed to identify ROIs in the diffusion metric map. In one example, an ROI can be defined as the entire transverse cord (i.e., a whole cord ROI). In other example, separate ROIs can be identified for gray matter (“GM”), dorsal column white matter (“DWM”), lateral column white matter (“LWM”), and ventral column white matter (“VWM”). When more than one ROI is selected in the diffusion metric map, the ROIs can be positioned symmetrically (e.g., with left-right symmetry) and the diffusion metrics in the symmetrically placed ROIs can be averaged for analysis.

As one example of the statistical analysis that can be performed to produce the imaging biomarker, a Student's t-test can be used to determine statistical significance between the diffusion metrics computed for the subject and established normal values. As mentioned above, the diffusion metrics can be analyzed in select ROIs within the first spinal cord level, or can be analyzed based on the whole spinal cord level.

As an example, for injuries that affect locomotor activities, the correlation between changes in diffusion metrics at the first spinal cord level and the tissue state at the second spinal cord level can be based on a correlation between an injury group and a BBB (“Basso, Beattie, and Bresnahan”) score using a Spearman correlation.

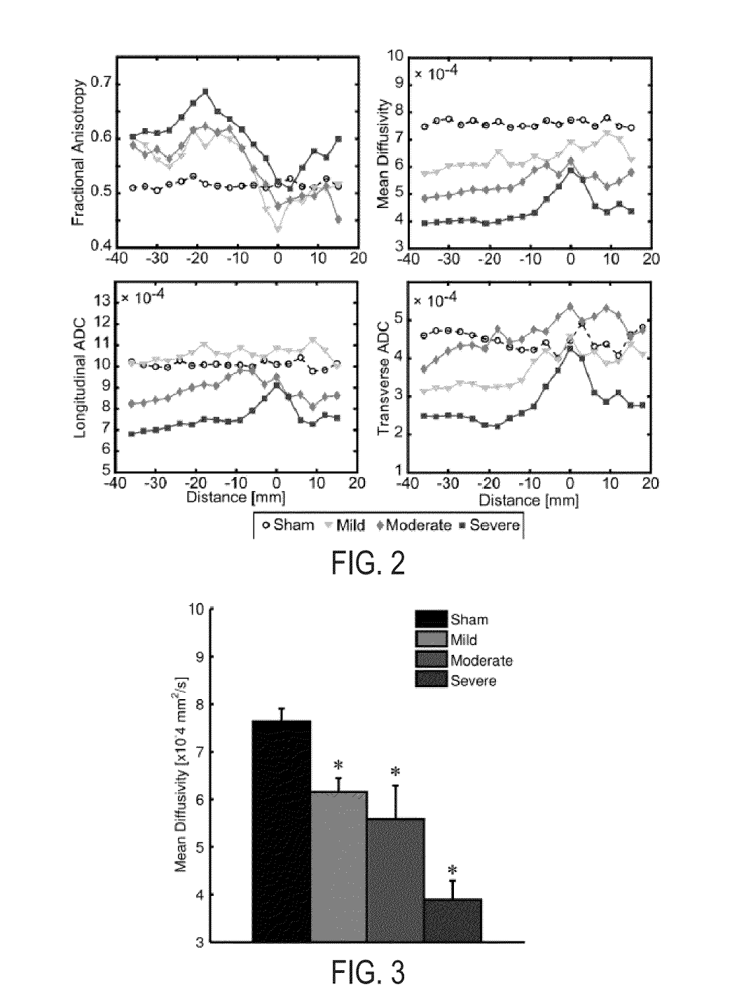

Implementations of the present invention have identified that there are variations in diffusion metric along the length of the spinal cord, with notable differences being present at the injury or pathology site. For instance, in the data presented in FIG. 2, diffusion metrics for each slice were analyzed from −40 mm rostral to an injury site to 20 mm caudal to an injury site in a cohort of clinical subjects.

In FIG. 2, an example comparison between severity groups rostral and caudal to the injury site is illustrated. Here, the injury site is located at 10 mm. FA, MD, lADC, and tADC metrics are shown for a whole cord ROI that includes white and gray matter. Spinal cord regions at and around the lesion site demonstrate drastically altered diffusion values than distal regions of the spinal cord. The clearest separation of diffusion values for each of the severity groups can be observed with the mean diffusivity metric. Visually, there is a clear separation between severity groups throughout the entire length of the cord, with the greatest difference occurring in mean diffusivity, which showed an 18% reduction for regions rostral to the injury site. This separation is also represented in FIG. 3, with mild, moderate, and severe injury groups being significantly different than the sham group when comparing average MD across the C1-C7 cervical segments.

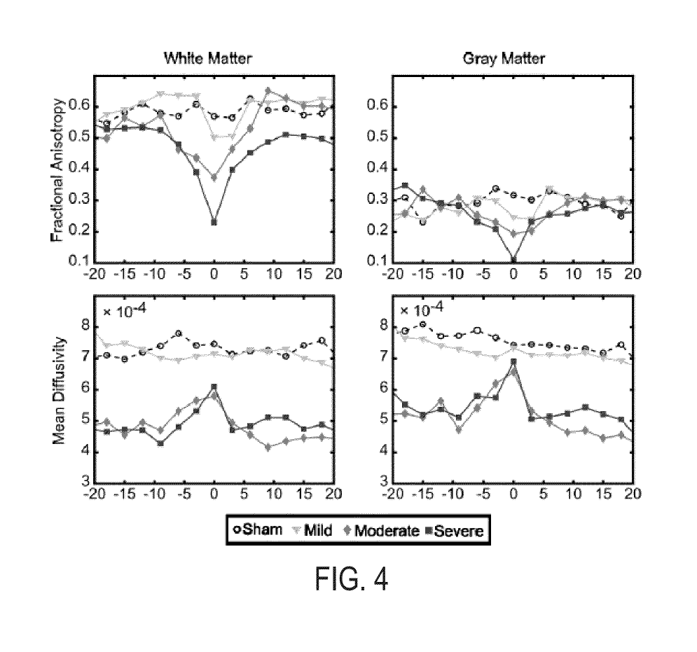

FIG. 4 illustrates a comparison of gray matter and white matter tracts. In this example, the separation between groups was consistent over the length of the cord for white matter tracts. The gray matter also showed separation based on severity, which was most visually apparent in MD and tADC.

The systems and methods of the present invention are thus capable of generating imaging biomarkers from DTI data that are sensitive enough to detect secondary injury distal to an injury or other pathology site in the spinal cord. As an example, these secondary injury effects can be manifested as a decrease in mean diffusivity in the cervical spinal cord, with the decrease in MD being proportional to the severity of injury. The imaging biomarker may also include a strong correlation between a diffusion metric, such as MD, measured at a distal level in the spinal cord and functional outcome, which in some embodiments can be measured by the BBB motor score. The generated imaging biomarkers are also sensitive enough to detect changes in both white matter and gray matter. For instance, as shown in FIG. 4, changes in diffusion metrics can be observed in both gray matter and individual tract ROIs.

Imaging biomarkers that are capable of indicating changes to the spinal cord at locations distal from an injury or pathological site have thus been provided. The imaging biomarkers are also capable of indicating a severity of the tissue state changes occurring at the injury of pathological site. These imaging biomarkers indicate that injury severity is associated with respective diffusion changes over the entire length of the spinal cord.

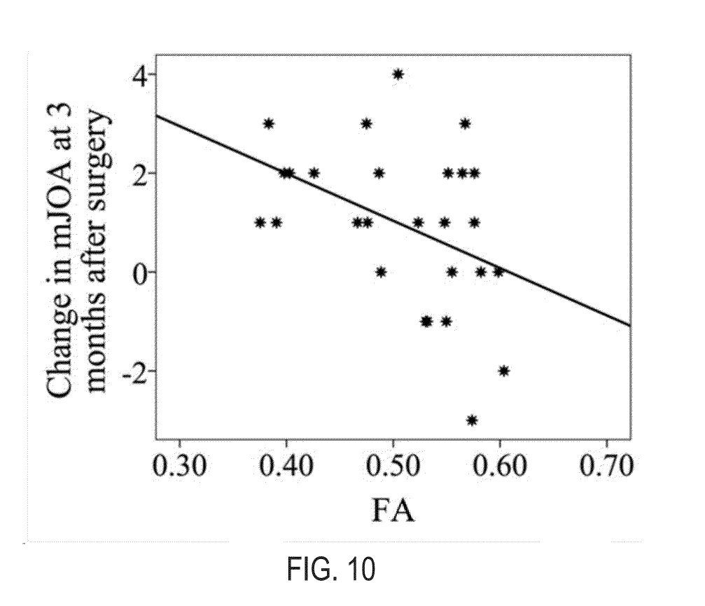

As one specific example, the method described above can be implemented to assess subjects with cervical myelopathies, such as cervical spondylotic myelopathy (“CSM”), which often results in a compression of the cervical spinal cord. As illustrated in FIG. 10, the diffusion properties of the spinal cord correlate with short-term clinical outcome in patients undergoing cervical spine surgery for CSM. For instance, fractional anisotropy and mean diffusivity can be associated with changes in clinical myelopathy score (e.g., the modified Japanese Orthopedic Association score) from before surgery to at least three months after surgery. It is contemplated that diffusion properties of the spinal cord will also correlate with preoperative clinical scores in CSM.

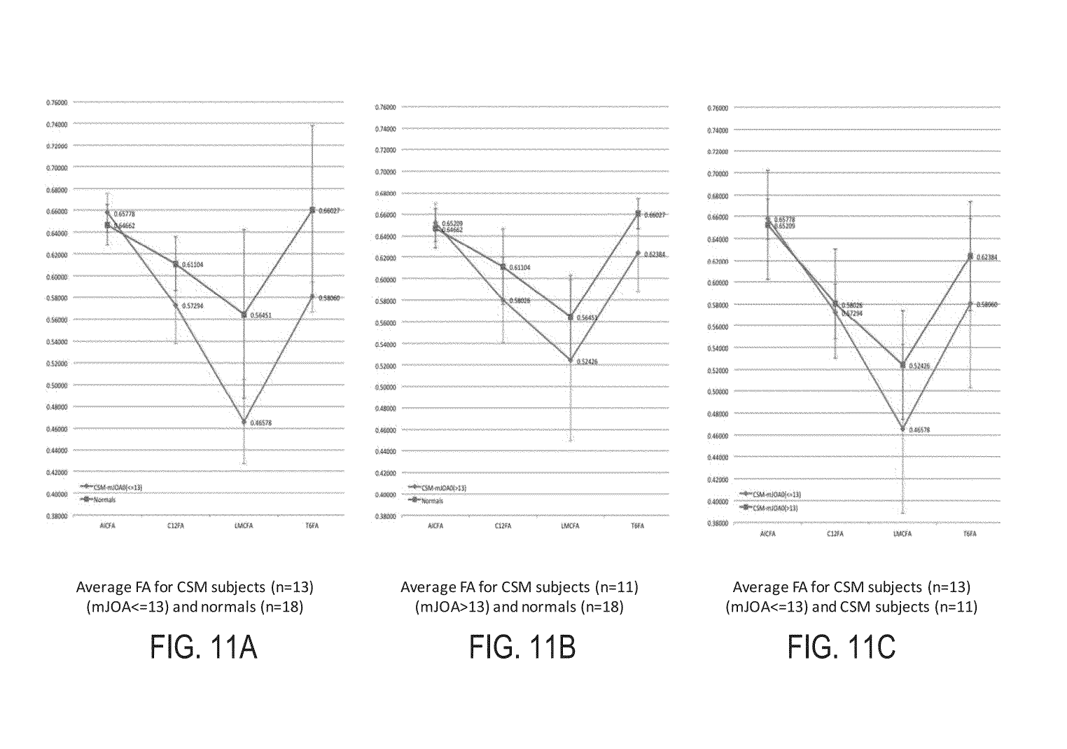

As described above, however, it may not always be possible or ideal to directly image the spinal cord at a region of compression. Thus, by implementing the method described above, the spinal cord can be imaged at a level that is distal to the compressed region and the diffusion metrics computed at that distal level can be correlated with diffusion metrics in the compressed region, which are then indicative of short-term clinical outcome as stated above. As illustrated in FIGS. 11A-11C, significant changes in the diffusion properties of the spinal cord can be measured along the entire extent of the spinal cord. For instance, the differences at C12, LMC, and T6 are significant, illustrating that the methods described here can be used to differentiate between “normal” and “pathology” across the entire extent of the spinal cord and not just at the most diseased level, which in this example is the level of maximum compression. Thus, the diffusion properties at the level of maximal compression (“LMC”) can be correlated or otherwise associated with diffusion properties measured at a spinal cord level distal to the LMC.

In some other embodiments, the imaging biomarker can be derived from a diffusion metric map at a spinal cord level, and can indicate a tissue state for tissues in the subject's brain. As an example, the imaging biomarker can indicate an injury to locations in the subject's brain based on the diffusion metrics computed at a spinal cord level in the subject's spinal cord. Such an imaging biomarker is advantageous for identifying injury to the subject's brain where direct imaging of the brain may not provide information that indicates the injury is present. For instance, the injury to the brain may be too subtle to identify through direct imaging.

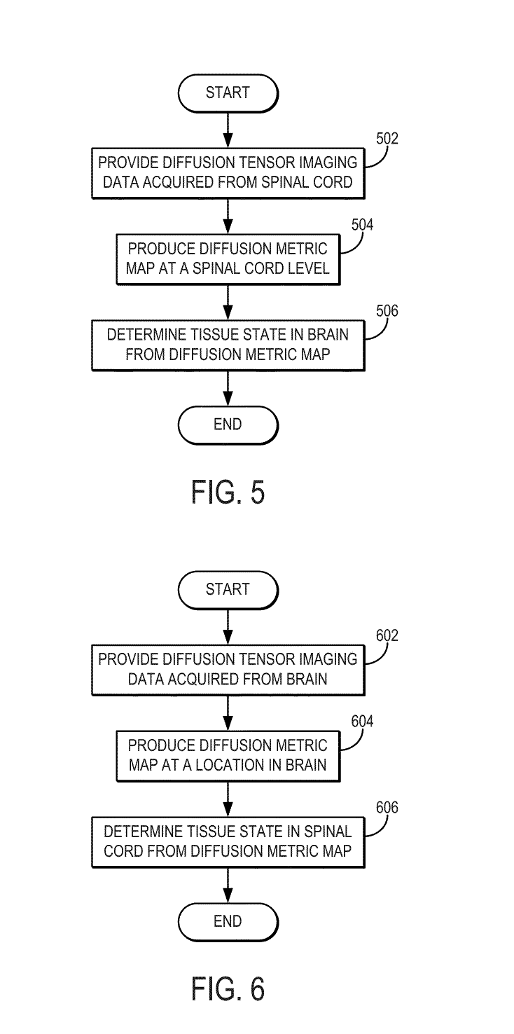

Referring now to FIG. 5, a flowchart setting forth the steps of an example method for producing an imaging biomarker that indicates a tissue state at a location in a subject's brain based on diffusion metrics computed at a location in the subject's spinal cord is illustrated.

The method includes providing DTI data that has been acquired from the subject's spinal cord with an MRI system, as indicated at step 502. As an example, the DTI data can be provided by retrieving previously acquired data from a storage device or database, or by acquiring the data using an MRI system. For the latter, the DTI data can be acquired by directing the MRI system to perform a suitable diffusion-weighted pulse sequence that is configured to acquire sufficient data for DTI. As an example, diffusion-weighted images can be acquired for six different diffusion encoding directions using a diffusion-weighted spin echo pulse sequence using b-values of 0 s/mm² for the reference image and a b-value of 500 s/mm² for the diffusion-weighted images.

The provided DTI data is then processed to produce at least one diffusion metric map at a spinal cord level, as indicated at step 504. This processing may include pre-processing steps, such as reorienting the diffusion-weighted image, and removing artifacts and distortions from the images. For example, the diffusion-weighted images can be co-registered to correct for-eddy current and susceptibility distortions.

Processing the DTI data includes computing a diffusion tensor for voxels associated with locations in the spinal cord location based on the DTI data acquired from those locations. From each computed diffusion tensor, one or more diffusion metrics can be computed, which can then be used to produce maps of each diffusion metric for the spinal cord level. As an example, the diffusion metric can be lADC, tADC, MD, or FA.

Based on the one or more diffusion metric maps produced at the spinal cord level, an imaging biomarker that indicates a tissue state for tissues at locations in the subject's brain is generated, as indicated at step 506. In general, the imaging biomarker is based on a statistical analysis of diffusion metrics at the spinal cord level. For instance, the diffusion metrics can be compared with established normal values and the comparison can be used to establish a correlation with a tissue state in the subject's brain.

In some embodiments, image analysis of the diffusion metric maps can be performed based on one or more ROIs selected or otherwise identified in the diffusion metric maps. For instance, a user can manually draw an ROI on the diffusion metric map, or an automated process can be performed to identify ROIs in the diffusion metric map. In one example, an ROI can be defined as the entire transverse cord (i.e., a whole cord ROI). In other example, separate ROIs can be identified for GM, DWM, LWM, or VWM. When more than one ROI is selected in the diffusion metric map, the ROIs can be positioned symmetrically (e.g., with left-right symmetry) and the diffusion metrics in the symmetrically placed ROIs can be averaged for analysis.

As one example of the statistical analysis that can be performed to produce the imaging biomarker, a Student's t-test can be used to determine statistical significance between the diffusion metrics computed for the subject and established normal values. As mentioned above, the diffusion metrics can be analyzed in select ROIs within the spinal cord level, or can be analyzed based on the whole spinal cord level.

As an example, for injuries that affect locomotor activities, the correlation between changes in diffusion metrics at the spinal cord level and the tissue state at locations in the brain can be based on a correlation between an injury group and a BBB (“Basso, Beattie, and Bresnahan”) score using a Spearman correlation.

It is a discovery of the invention that the imaging biomarkers discussed above are capable of detecting the subtle structural changes that occur in the brain following a spinal cord injury, or in relation to a pathology in the spinal cord. As discussed below, in some other embodiments, the imaging biomarkers are also capable of detecting subtle changes that occur in the spinal cord following a traumatic brain injury, or in relation to a pathology in the brain.

Table 1 illustrates data from an experiment in which rat brains with a sham, mild, moderate, or severe contusion spinal injury were scanned ex vivo ten weeks after injury in order to characterize how internal structures of the brain vary with injury severity. Automatic voxel-based statistical analysis was completed and verified through manual selection of ROIs. Following DTI, histological analysis verified structural locations associated with imaging results. Diffusion indices indicated structural changes to the corticospinal tract (“CS”) and internal capsule (“IC”) regions in the brain and brainstem that correlated with a decrease in motor function associated with injury severity.

TABLE 1

| | Sham | Mild | Moderate | Severe |
|---|---|---|---|---|
| FA (IC) | 0.304 (±0.05) | 0.348 (±0.06) | 0.352 (±0.11)* | 0.395 (±0.07)*** |
| MD (IC) | 1.131 (±0.03 | 1.099 (±0.02)* | 1.074 (±0.06)* | 1.076 (±0.01)** |
| FA (CS) | 0.424 (±0.03) | 0.529 (±0.11) | 0.550 (±0.08) | 0.574 (±0.09) |
| MD (CS) | 1.260 (±0.01) | 1.058 (±0.01)* | 1.045 (±0.00)** | 0.950 (±0.00)** |

*p < 0.05
**p < 0.01
***p < 0.001

In some other embodiments, the imaging biomarker can be derived from a diffusion metric map at a location in the subject's brain, and can indicate a tissue state for tissues in the subject's spinal cord. As an example, the imaging biomarker can indicate an injury to tissue at one or more spinal cord levels in the subject's spinal cord based on the diffusion metrics computed at a location in the subject's brain. Such an imaging biomarker is advantageous for identifying injury to the subject's spinal cord that is correlated with an injury to the subject's brain.

Referring now to FIG. 6, a flowchart setting forth the steps of an example method for producing an imaging biomarker that indicates a tissue state at a spinal cord level in a subject's spinal cord based on diffusion metrics computed at a location in the subject's brain is illustrated.

The method includes providing DTI data that has been acquired from the subject's brain with an MRI system, as indicated at step 602. As an example, the DTI data can be provided by retrieving previously acquired data from a storage device or database, or by acquiring the data using an MRI system. For the latter, the DTI data can be acquired by directing the MRI system to perform a suitable diffusion-weighted pulse sequence that is configured to acquire sufficient data for DTI. As an example, diffusion-weighted images can be acquired for six different diffusion encoding directions using a diffusion-weighted spin echo pulse sequence using b-values of 0 s/mm² for the reference image and a b-value of 500 s/mm² for the diffusion-weighted images.

The provided DTI data is then processed to produce at least one diffusion metric map at a location in the subject's brain, as indicated at step 604. This processing may include pre-processing steps, such as reorienting the diffusion-weighted image, and removing artifacts and distortions from the images. For example, the diffusion-weighted images can be co-registered to correct for-eddy current and susceptibility distortions. As an example, the location in the subject's brain can include a slice location, such as an axial, sagittal, coronal, or oblique slice location. As another example, the location in the subject's brain can include a volume-of-interest, such as a volume-of-interest that covers some or all of the subject's brain.

Processing the DTI data includes computing a diffusion tensor for voxels associated with locations in the subject's brain based on the DTI data acquired from those locations. From each computed diffusion tensor, one or more diffusion metrics can be computed, which can then be used to produce maps of each diffusion metric for the spinal cord level. As an example, the diffusion metric can be lADC, tADC, MD, or FA.

Based on the one or more diffusion metric maps produced for locations in the subject's brain, an imaging biomarker that indicates a tissue state for tissues at locations in a spinal cord level of the subject's spinal cord is generated, as indicated at step 606. In general, the imaging biomarker is based on a statistical analysis of diffusion metrics in the subject's brain. For instance, the diffusion metrics can be compared with established normal values and the comparison can be used to establish a correlation with a tissue state in the subject's spinal cord. It is a discovery of the invention that diffusion metrics computed for locations in the subject's brain can be correlated with the tissue state at multiple different spinal cord levels in the spinal cord. Thus, the diffusion metrics in the subject's brain can be used as an imaging biomarker for the tissue state of tissues throughout the subject's spinal cord.

In some embodiments, image analysis of the diffusion metric maps can be performed based on one or more ROIs selected or otherwise identified in the diffusion metric maps. For instance, a user can manually draw an ROI on the diffusion metric map, or an automated process can be performed to identify ROIs in the diffusion metric map. In one example, an ROI can be defined as the entire slice locations, or as the entire brain volume. When more than one ROI is selected in the diffusion metric map, the ROIs can be positioned symmetrically (e.g., with left-right symmetry) and the diffusion metrics in the symmetrically placed ROIs can be averaged for analysis.

As one example of the statistical analysis that can be performed to produce the imaging biomarker, a Student's t-test can be used to determine statistical significance between the diffusion metrics computed for the subject and established normal values. As mentioned above, the diffusion metrics can be analyzed in select ROIs within the subject's brain, or can be analyzed based on a volume-of-interest that may encompass the subject's whole brain.

As an example, for injuries that affect locomotor activities, the correlation between changes in diffusion metrics in the subject's brain and the tissue state at locations in one or more spinal cord levels can be based on a correlation between an injury group and a BBB (“Basso, Beattie, and Bresnahan”) score using a Spearman correlation.

The current neuroimaging techniques to monitor the progression of neuronal stem cells are limited. Within animal models, end point histology is often used as a means for determining progress of transplant injections. Removing tissue samples is invasive and is not a feasible option for humans, nevertheless there has been development of non-invasive imaging techniques that track stem cells. However, in these examples the magnetic tracer can produce susceptibility blooming which can degrade the MR image. The contrast agents that are used can also cause harm to renal function.

There have been a few studies where changes in water diffusion reflect axonal regeneration following the transplantation of fibroblasts or autoimmune T-cells. The changes in axonal structure found through variations in water diffusion from these cell types suggest that neuronal stem cells that differentiate into cells specific to the central nervous system will impact the diffusion of water in a similar way.

In some other embodiments, the imaging biomarker can be derived from a diffusion metric map at a first spinal cord level, and can indicate an efficacy of a spinal cord treatment in tissues at a second spinal cord level in the subject's spinal cord. As an example, the spinal cord treatment may include a stem cell therapy. The imaging biomarker can thus indicate an efficacy of treatment in tissues at the second spinal cord level based on the diffusion metrics computed at the first spinal cord level. In some instances, the first spinal cord level and the second spinal cord level can be the same spinal cord level.

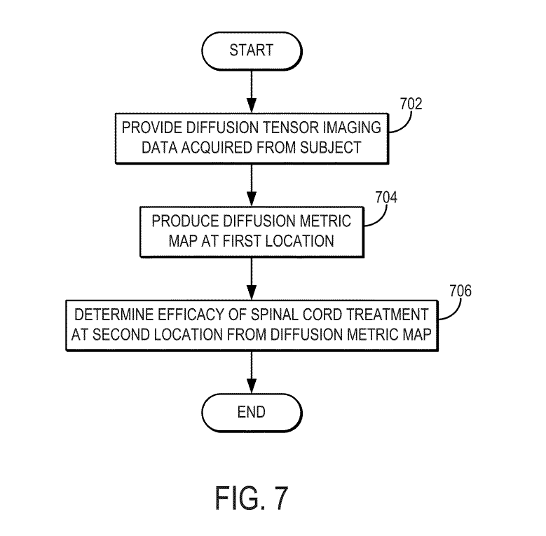

Referring now to FIG. 7, a flowchart setting forth the steps of an example method for producing an imaging biomarker that indicates an efficacy of a treatment delivered to a subject's central nervous system based on diffusion metrics computed at a location in the subject's spinal cord is illustrated.

The method includes providing DTI data that has been acquired from the subject's spinal cord with an MRI system, as indicated at step 702. As an example, the DTI data can be provided by retrieving previously acquired data from a storage device or database, or by acquiring the data using an MRI system. For the latter, the DTI data can be acquired by directing the MRI system to perform a suitable diffusion-weighted pulse sequence that is configured to acquire sufficient data for DTI. As an example, diffusion-weighted images can be acquired for six different diffusion encoding directions using a diffusion-weighted spin echo pulse sequence using b-values of 0 s/mm² for the reference image and a b-value of 500 s/mm² for the diffusion-weighted images.

The provided DTI data is then processed to produce at least one diffusion metric map at a first spinal cord level, as indicated at step 704. This processing may include pre-processing steps, such as reorienting the diffusion-weighted image, and removing artifacts and distortions from the images. For example, the diffusion-weighted images can be co-registered to correct for-eddy current and susceptibility distortions.

Processing the DTI data includes computing a diffusion tensor for voxels associated with locations in the spinal cord location based on the DTI data acquired from those locations. From each computed diffusion tensor, one or more diffusion metrics can be computed, which can then be used to produce maps of each diffusion metric for the spinal cord level. As an example, the diffusion metric can be lADC, tADC, MD, or FA.

Based on the one or more diffusion metric maps produced at the first spinal cord level, an imaging biomarker that indicates a tissue state for tissues at a second spinal cord level is generated, as indicated at step 706. In general, the imaging biomarker is based on a statistical analysis of diffusion metrics at the first spinal cord level. For instance, the diffusion metrics can be compared with established normal values and the comparison can be used to establish a correlation with a tissue state in the second spinal cord level. As an example, the tissue state can indicate an efficacy of a treatment delivered to the subject's spinal cord, such as a stem cell-based treatment.

In some embodiments, image analysis of the diffusion metric maps can be performed based on one or more ROIs selected or otherwise identified in the diffusion metric maps. For instance, a user can manually draw an ROI on the diffusion metric map, or an automated process can be performed to identify ROIs in the diffusion metric map. In one example, an ROI can be defined as the entire transverse cord (i.e., a whole cord ROI). In other example, separate ROIs can be identified for GM, DWM, LWM, or VWM. When more than one ROI is selected in the diffusion metric map, the ROIs can be positioned symmetrically (e.g., with left-right symmetry) and the diffusion metrics in the symmetrically placed ROIs can be averaged for analysis.

As one example of the statistical analysis that can be performed to produce the imaging biomarker, a Student's t-test can be used to determine statistical significance between the diffusion metrics computed for the subject and established normal values. As mentioned above, the diffusion metrics can be analyzed in select ROIs within the first spinal cord level, or can be analyzed based on the whole spinal cord level.

As an example, for injuries that affect locomotor activities, the correlation between changes in diffusion metrics at the first spinal cord level and the tissue state at the second spinal cord level can be based on a correlation between an injury group and a BBB (“Basso, Beattie, and Bresnahan”) score using a Spearman correlation.

It will be appreciated that the imaging biomarkers discussed above can be generated and tracked over a period of time, whether days, weeks, months, or years, to monitor the efficacy of a treatment provided to a subject's spinal cord.

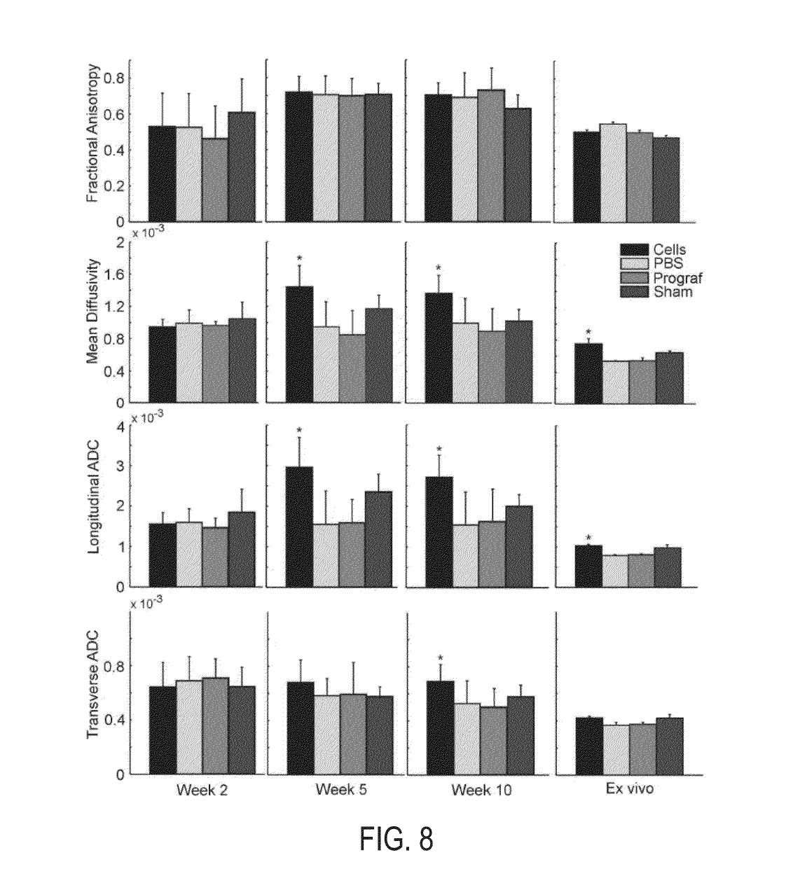

FIG. 8 illustrates data from an experiment in which diffusion metrics were measured in rats having a spinal contusion injury. Rats were randomized into four groups to receive no transplant injection (n=10, sham group), sterile phosphate buffered saline (PBS, n=10), sterile PBS with 50 mg/kg of the immunosuppressant drug Tacrolimus (Prograf; n=10; Mylan Pharmaceuticals Inc; Canonsburg, Pa.), or C17.2 neural stem cells (“NSCs”) with Prograf (n=10).

The diffusion metrics were measured in ROIs that encompassed transverse slices through the spinal cord. Rats that received the NSC C17.2 transplant showed significantly different diffusion values compared to the control groups (p<0.05) in multiple time points for MD, lADC, and tADC. Fractional anisotropy for all time points did not indicate that the cell line was significantly different than the control groups (p>0.05). Mean diffusivity showed that at weeks 5 and 10, and in ex vivo scans that there was a significant increase in the diffusion of water when compared to the control groups (p<0.05). The diffusion in the longitudinal direction (e.g., measured by lADC) was also significantly greater in weeks 5, 10, and ex vivo time points (p<0.05). The diffusion of water through the cord became significantly greater for the rats that received the C17.2 line than the control rats at week 10 and held a significant result for the ex vivo scans (p<0.05).

The results from this study suggest that the imaging biomarkers described here are capable of non-invasively observing the effects of cellular transplants in the spinal cord. Using a non-invasive technique that can monitor migration of the stem cells and suggest what structural effects could be occurring from the stem cells provides significant clinical implications in the treatment of SCI.

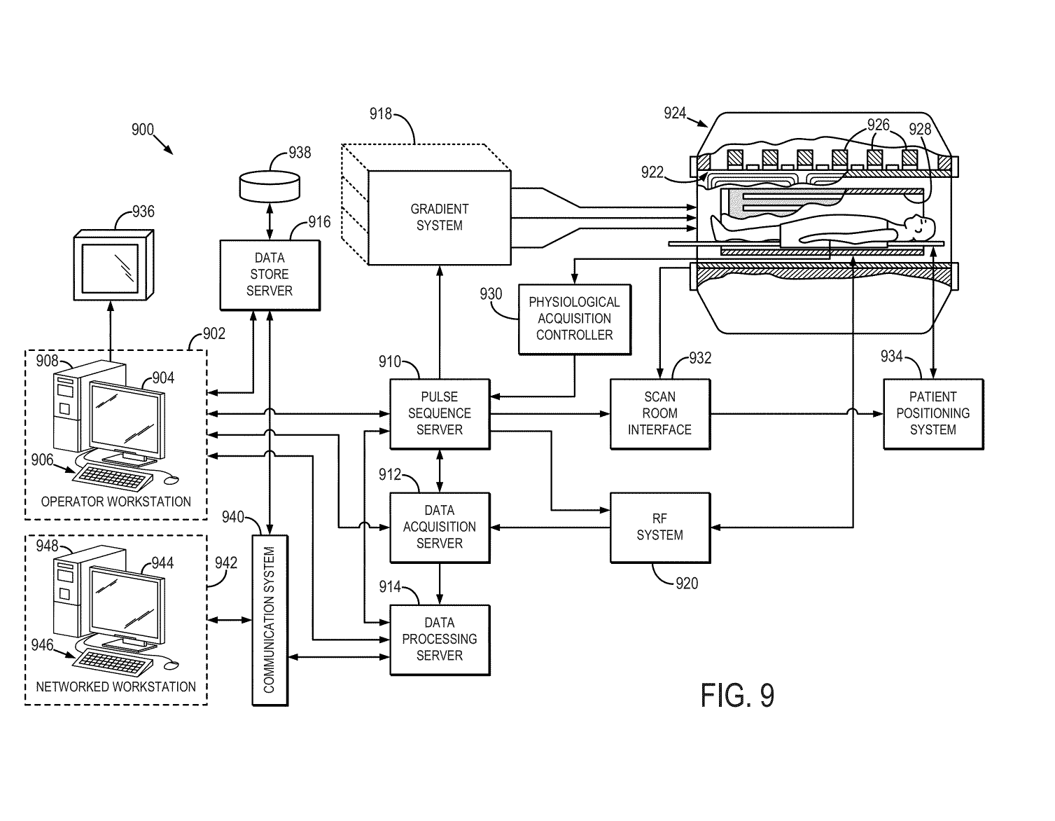

Referring particularly now to FIG. 9, an example of a magnetic resonance imaging (“MRI”) system 900 is illustrated. The MRI system 900 includes an operator workstation 902, which will typically include a display 904; one or more input devices 906, such as a keyboard and mouse; and a processor 908. The processor 908 may include a commercially available programmable machine running a commercially available operating system. The operator workstation 902 provides the operator interface that enables scan prescriptions to be entered into the MRI system 900. In general, the operator workstation 902 may be coupled to four servers: a pulse sequence server 910; a data acquisition server 912; a data processing server 914; and a data store server 916. The operator workstation 902 and each server 910, 912, 914, and 916 are connected to communicate with each other. For example, the servers 910, 912, 914, and 916 may be connected via a communication system 940, which may include any suitable network connection, whether wired, wireless, or a combination of both. As an example, the communication system 940 may include both proprietary or dedicated networks, as well as open networks, such as the internet.

The pulse sequence server 910 functions in response to instructions downloaded from the operator workstation 902 to operate a gradient system 918 and a radiofrequency (“RF”) system 920. Gradient waveforms necessary to perform the prescribed scan are produced and applied to the gradient system 918, which excites gradient coils in an assembly 922 to produce the magnetic field gradients Gₓ, G_{y}, and G_{z} used for position encoding magnetic resonance signals. The gradient coil assembly 922 forms part of a magnet assembly 924 that includes a polarizing magnet 926 and a whole-body RF coil 928.

RF waveforms are applied by the RF system 920 to the RF coil 928, or a separate local coil (not shown in FIG. 9), in order to perform the prescribed magnetic resonance pulse sequence. Responsive magnetic resonance signals detected by the RF coil 928, or a separate local coil (not shown in FIG. 9), are received by the RF system 920, where they are amplified, demodulated, filtered, and digitized under direction of commands produced by the pulse sequence server 910. The RF system 920 includes an RF transmitter for producing a wide variety of RF pulses used in MRI pulse sequences. The RF transmitter is responsive to the scan prescription and direction from the pulse sequence server 910 to produce RF pulses of the desired frequency, phase, and pulse amplitude waveform. The generated RF pulses may be applied to the whole-body RF coil 928 or to one or more local coils or coil arrays (not shown in FIG. 9).

The RF system 920 also includes one or more RF receiver channels. Each RF receiver channel includes an RF preamplifier that amplifies the magnetic resonance signal received by the coil 928 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 910 also optionally receives patient data from a physiological acquisition controller 930. By way of example, the physiological acquisition controller 930 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 910 to synchronize, or “gate,” the performance of the scan with the subject's heart beat or respiration.

The pulse sequence server 910 also connects to a scan room interface circuit 932 that receives signals from various sensors associated with the condition of the patient and the magnet system. It is also through the scan room interface circuit 932 that a patient positioning system 934 receives commands to move the patient to desired positions during the scan.

The digitized magnetic resonance signal samples produced by the RF system 920 are received by the data acquisition server 912. The data acquisition server 912 operates in response to instructions downloaded from the operator workstation 902 to receive the real-time magnetic resonance data and provide buffer storage, such that no data is lost by data overrun. In some scans, the data acquisition server 912 does little more than pass the acquired magnetic resonance data to the data processor server 914. However, in scans that require information derived from acquired magnetic resonance data to control the further performance of the scan, the data acquisition server 912 is programmed to produce such information and convey it to the pulse sequence server 910. For example, during prescans, magnetic resonance data is acquired and used to calibrate the pulse sequence performed by the pulse sequence server 910. As another example, navigator signals may be acquired and used to adjust the operating parameters of the RF system 920 or the gradient system 918, or to control the view order in which k-space is sampled. In still another example, the data acquisition server 912 may also be employed to process magnetic resonance signals used to detect the arrival of a contrast agent in a magnetic resonance angiography (“MRA”) scan. By way of example, the data acquisition server 912 acquires magnetic resonance data and processes it in real-time to produce information that is used to control the scan.

The data processing server 914 receives magnetic resonance data from the data acquisition server 912 and processes it in accordance with instructions downloaded from the operator workstation 902. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 914 are conveyed back to the operator workstation 902 where they are stored. Real-time images are stored in a data base memory cache (not shown in FIG. 9), from which they may be output to operator display 912 or a display 936 that is located near the magnet assembly 924 for use by attending physicians. Batch mode images or selected real time images are stored in a host database on disc storage 938. When such images have been reconstructed and transferred to storage, the data processing server 914 notifies the data store server 916 on the operator workstation 902. The operator workstation 902 may be used by an operator to archive the images, produce films, or send the images via a network to other facilities.

The MRI system 900 may also include one or more networked workstations 942. By way of example, a networked workstation 942 may include a display 944; one or more input devices 946, such as a keyboard and mouse; and a processor 948. The networked workstation 942 may be located within the same facility as the operator workstation 902, or in a different facility, such as a different healthcare institution or clinic.

The networked workstation 942, whether within the same facility or in a different facility as the operator workstation 902, may gain remote access to the data processing server 914 or data store server 916 via the communication system 940. Accordingly, multiple networked workstations 942 may have access to the data processing server 914 and the data store server 916. In this manner, magnetic resonance data, reconstructed images, or other data may exchanged between the data processing server 914 or the data store server 916 and the networked workstations 942, such that the data or images may be remotely processed by a networked workstation 942. This data may be exchanged in any suitable format, such as in accordance with the transmission control protocol (“TCP”), the internet protocol (“IP”), or other known or suitable protocols.

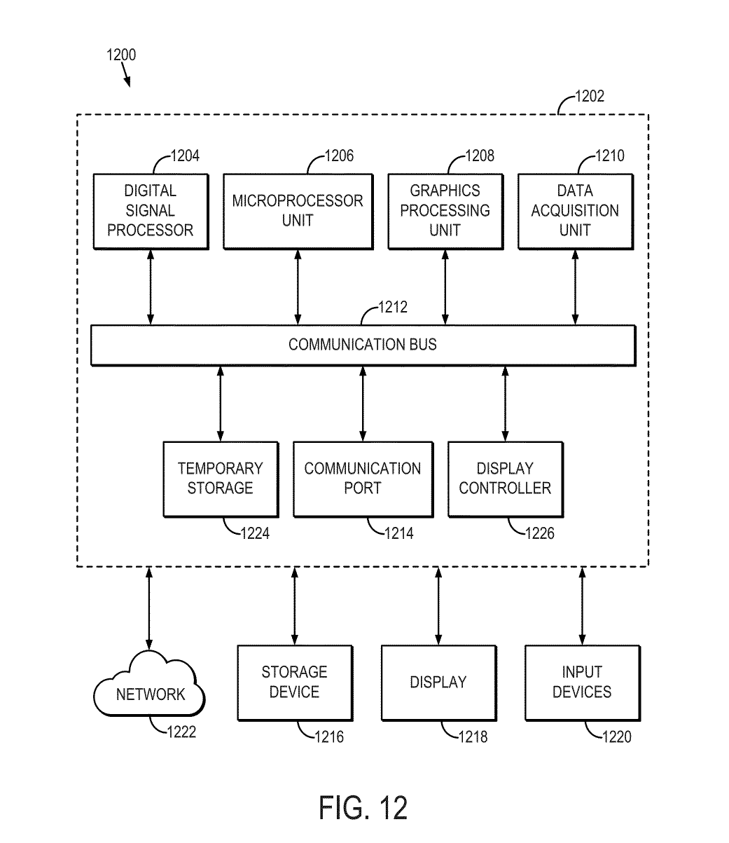

Referring now to FIG. 12, a block diagram of an example computer system 1200 that can be configured to compute diffusion metrics and correlate those values to the state of tissues at different locations in the central nervous system, as described above, is illustrated. The magnetic resonance images from which the diffusion metrics are computed can be provided to the computer system 1200 from the respective MRI system and received in a processing unit 1202.

In some embodiments, the processing unit 1202 can include one or more processors. As an example, the processing unit 1202 may include one or more of a digital signal processor (“DSP”) 1204, a microprocessor unit (“MPU”) 1206, and a graphics processing unit (“GPU”) 1208. The processing unit 1202 can also include a data acquisition unit 1210 that is configured to electronically receive data to be processed, which may include magnetic resonance image data, magnetic resonance image images, or diffusion metric maps. The DSP 1204, MPU 1206, GPU 1208, and data acquisition unit 1210 are all coupled to a communication bus 1212. As an example, the communication bus 1212 can be a group of wires, or a hardwire used for switching data between the peripherals or between any component in the processing unit 1202.

The DSP 1204 can be configured to receive and processes the magnetic resonance image data, magnetic resonance image images, or diffusion metric maps. The MPU 1206 and GPU 1208 can also be configured to process the magnetic resonance image data, magnetic resonance image images, or diffusion metric maps in conjunction with the DSP 1204. As an example, the MPU 1206 can be configured to control the operation of components in the processing unit 1202 and can include instructions to perform processing of the magnetic resonance image data, magnetic resonance image images, or diffusion metric maps on the DSP 1204. Also as an example, the GPU 1208 can process image graphics.

In some embodiments, the DSP 1204 can be configured to process the magnetic resonance image data, magnetic resonance image images, or diffusion metric maps received by the processing unit 1202 in accordance with the algorithms described above. Thus, the DSP 1204 can be configured to compute diffusion metrics and correlate diffusion metrics at a first location in the central nervous system to a tissue state at a second location in the central nervous system.

The processing unit 1202 preferably includes a communication port 1214 in electronic communication with other devices, which may include a storage device 1216, a display 1218, and one or more input devices 1220. Examples of an input device 1220 include, but are not limited to, a keyboard, a mouse, and a touch screen through which a user can provide an input.

The storage device 1216 is configured to store images, whether provided to or processed by the processing unit 1202. The display 1218 is used to display images, such as images that may be stored in the storage device 1216, and other information. Thus, in some embodiments, the storage device 1216 and the display 1218 can be used for displaying diffusion metric maps or reports that indicate a correlation of diffusion metrics with clinical outcome, such as a postoperative clinical outcome in subjects with CSM, which may include data plots or other reports based on a correlation process.

The processing unit 1202 can also be in electronic communication with a network 1222 to transmit and receive data, including CT images, MR images, and other information. The communication port 1214 can also be coupled to the processing unit 1202 through a switched central resource, for example the communication bus 1212.

The processing unit 1202 can also include a temporary storage 1224 and a display controller 1226. As an example, the temporary storage 1224 can store temporary information. For instance, the temporary storage 1224 can be a random access memory.

The present invention has been described in terms of one or more preferred embodiments, and it should be appreciated that many equivalents, alternatives, variations, and modifications, aside from those expressly stated, are possible and within the scope of the invention.

1. A method for producing an imaging biomarker that indicates a tissue state at a spinal cord level using a magnetic resonance imaging (MRI) system, the steps of the method comprising:
(a) providing diffusion tensor imaging data acquired from a spinal cord of a subject with an MRI system;
(b) producing a diffusion metric map at a first spinal cord level from the provided diffusion tensor imaging data, wherein the diffusion metric map contains diffusion metric values associated with locations in the first spinal cord level;
(c) determining, based on the produced diffusion metric map at the first spinal cord level, a tissue state for tissues at a second spinal cord level that is distal to the first spinal cord level.

2. The method as recited in claim 1, wherein the second spinal cord level is rostral to the first spinal cord level.

3. The method as recited in claim 1, wherein the second spinal cord level is caudal to the first spinal cord level.

4. The method as recited in claim 1, wherein step (c) includes comparing the diffusion metric values in the diffusion metric map to established normal diffusion metric values.

5. The method as recited in claim 4, wherein step (c) includes comparing the diffusion metric values in at least one selected region-of-interest of the diffusion metric map to the established normal diffusion metric values.

6. The method as recited in claim 1, wherein the diffusion metric map values are at least one of longitudinal apparent diffusion coefficient, transverse apparent diffusion coefficient, mean diffusivity, or fractional anisotropy.

7. The method as recited in claim 1, further comprising generating a report that indicates a correlation between the tissue state for tissues at the second spinal cord level and a clinical outcome.

8. The method as recited in claim 7, wherein the clinical outcome is a clinical outcome associated with cervical spondylotic myelopathy.

9. The method as recited in claim 8, wherein the clinical outcome is a short-term postoperative clinical outcome.

10. A method for producing an imaging biomarker that indicates a tissue state at locations in a brain of a subject using a magnetic resonance imaging (MRI) system, the steps of the method comprising:
(a) providing diffusion tensor imaging data acquired from a spinal cord of a subject with an MRI system;
(b) producing a diffusion metric map at a spinal cord level from the provided diffusion tensor imaging data, wherein the diffusion metric map contains diffusion metric values associated with locations in the spinal cord level;
(c) determining, based on the produced diffusion metric map at the spinal cord level, a tissue state for tissues in a brain of the subject.

11. A method for producing an imaging biomarker that indicates a tissue state at a spinal cord level using a magnetic resonance imaging (MRI) system, the steps of the method comprising:
(a) providing diffusion tensor imaging data acquired from a brain of a subject with an MRI system;
(b) producing a diffusion metric map at a location in the subject's brain from the provided diffusion tensor imaging data, wherein the diffusion metric map contains diffusion metric values associated with locations in the subject's brain;
(c) determining, based on the produced diffusion metric map at the location in the subject's brain, a tissue state for tissues at the spinal cord level in the subject's spinal cord.

12. A method for producing an imaging biomarker that indicates an efficacy of a treatment delivered to a spinal cord of a subject, the steps of the method comprising:
(a) providing diffusion tensor imaging data acquired from a location in a central nervous system (CNS) of a subject with a magnetic resonance imaging system (MRI);
(b) producing a diffusion metric map at the location in the subject's CNS from the provided diffusion tensor imaging data, wherein the diffusion metric map contains diffusion metric values associated with locations in the location in the subject's CNS;
(c) determining, based on the produced diffusion metric map at the first location, an efficacy of a spinal cord treatment at a location in a spinal cord of the subject.

13. The method as recited in claim 9, wherein the location in the subject's CNS is a brain of the subject.

14. The method as recited in claim 9, wherein the location in the subject's CNS is a first spinal cord level in the subject's spinal cord, and the location in the subject's spinal cord at which the efficacy of the spinal cord treatment is determined in step (c) is different from the first spinal cord level.
